SEQUENCE LISTING (21 sequences)

SEQ ID NO: 1 - DNA, 293 bp, Streptococcus agalactiae
gacaggcattatgaggtatattttgcgactcatcggcagatgtaaaatgccagttaaatataactgcaaaaaatacaaattcttacgcattagctgcctaaaaaacagcctgcgtgatcttcacaagattgtttgcgttttgctagaaggtcttatttatcagcaaactacgtttggctactgtctagttagttaaaaagagatttatagactcgctatgtgagggcttgagttatgtgtcatcacctagttaaatcaatacataacctatagttgtagacaaatatattagc

SEQ ID NO: 2 - DNA, 18 bp, Streptococcus agalactiae
gacaggcattatgaggta

SEQ ID NO: 3 - DNA, 21 bp, Streptococcus agalactiae
gctaatatatttgtctacaac

SEQ ID NO: 4 - DNA, 23 bp, Streptococcus agalactiae
  misc_feature (23)..(23): Residue 23 labelled with a fluorescein group
ttgcgttttgctagaaggtctta

SEQ ID NO: 5 - DNA, 22 bp, Streptococcus agalactiae
  misc_feature (1)..(1): Residue 1 is labelled with a LightCycler Red fluorophore (LightCycler Red 640)
tatcagcaaactacgtttggct

SEQ ID NO: 6 - DNA, 35 bp, Streptococcus agalactiae
gacaggcattatgaggtaatacccaacttggaatg

SEQ ID NO: 7 - DNA, 41 bp, Streptococcus agalactiae
gctaatatatttgtctacaactcttcaccagaataaaattg

SEQ ID NO: 8 - DNA, 18 bp, Streptococcus agalactiae
ggggmytacggwttcgac

SEQ ID NO: 9 - DNA, 18 bp, Streptococcus agalactiae
gggartcgaaccrsgtcc

SEQ ID NO: 10 - DNA, 17 bp, Streptococcus agalactiae
  misc_feature (17)..(17): Residue 17 is labelled with a fluorescein group
tgaatgtatcccctgga

SEQ ID NO: 11 - DNA, 19 bp, Streptococcus agalactiae
  misc_feature (1)..(1): Residue 1 is labelled with a LightCycler Red fluorphore (LightCycler Red 705)
tggcactggtaccatctaa

SEQ ID NO: 12 - DNA, 334 bp, Streptococcus agalactiae
ggggmttacggwttcgacaggcattatgaggtatattttgcgactcatcggcagatgtaaaatgccagttaaatataactgcaaaaaatacaaattcttacgcattagctgcctaaaaaacagcctgcgtgatcttcacaagattgtttgcgttttgctagaaggtcttatttatcagcaaactacgtttggctactgtctagttagttaaaaagagatttatagactcgctatgtgagggcttgagttatgtgtcatcacctagttaaatcaatacataacctatagttgtagacaaatatattagcagatgtttggaccyggttcgaytccc

SEQ ID NO: 13 - DNA, 334 bp, Streptococcus agalactiae
ggggmttacggwttcgacaggcattatgaggtatatttcgcgactcatcggcagatgtaaaatgccagttaaatataactgcaaaaaatacaaattcttacgcattagctgcctaaaaaacagcctgcgtgatcttcacaagattgtttgcgttttgctagaaggtcttatttatcagcaaactacgtttggctactgtctagttagttaaaaagagatttatagactcactatgtgagggcttgagttatgtgtcatcacctagttaaatcaatacataacctatagttgtagacaaatatattagcagatgtttggacsyggttcgaytccc

SEQ ID NO: 14 - DNA, 334 bp, Streptococcus agalactiae
ggggmttacggwttcgacaggcattatgaggtatatttcgcgactcatcggcagatgtaaaatgccagttaaatataactgcaaaaaatacaaattcttacgcattagctgcctaaaaaacagcctgcgtgatcttcacaagattgtttgcgttttgctagaaggtcttatttatcagcaaactacgtttggctactgtctagttagttaaaaagagatttatagactcgctatgtgagggcttgagttatgtgtcatcacctagttaaatcaatacataacctatagttgtagacaaatatattagcagatgtttggacsyggttcgaytccc

SEQ ID NO: 15 - DNA, 334 bp, Streptococcus agalactiae
ggggmttacggwttcgacaggcattatgaggtatatttcgcgactcatcggcagatgtaaaatgccagttaaatataactgcaaaaaatacaaattcttacgcattagctgcctaaaaaacagcctgcgtgatcttcacaagattgtttgcgttttgctagaaggtcttatttatcagcaaactacgtttggctactgtctagttagttaaaaagagatttatagactcgctatgtgagggcttgagttatgtgtcatcacctagttaaatcaatacataacctatagttgtagacaaatatattagcagatgtttggaccyggttcgaytccc

SEQ ID NO: 16 - DNA, 334 bp, Streptococcus agalactiae; identical to SEQ ID NO: 12

SEQ ID NO: 17 - DNA, 334 bp, Streptococcus agalactiae; identical to SEQ ID NO: 15

SEQ ID NO: 18 - DNA, 334 bp, Streptococcus agalactiae; identical to SEQ ID NO: 12

SEQ ID NO: 19 - DNA, 334 bp, Streptococcus agalactiae; identical to SEQ ID NO: 15

SEQ ID NO: 20 - DNA, 334 bp, Streptococcus agalactiae; identical to SEQ ID NO: 12

SEQ ID NO: 21 - DNA, 334 bp, Streptococcus agalactiae; identical to SEQ ID NO: 12

(19) United States
(12) Patent Application Publication
(10) Pub. No.: US 2009/0246764 A1
(43) Pub. Date: Oct. 1, 2009

(54) Method for the detection of group B Streptococcus (GBS) (Streptococcus agalactiae) in mammals

(75) Inventors: Thomas Gerard Barry, Kinvara (IE); Majella Mary Maher, Moycullen (IE); Terence James Smith, Galway (IE)

Correspondence Address: BIRCH STEWART KOLASCH & BIRCH, PO BOX 747, FALLS CHURCH, VA, 22040-0747, US

(73) Assignee: NATIONAL UNIVERSITY OF IRELAND GALWAY, Galway (IE)

(21) Appl. No.: 12/078,432

(22) Filed: Mar. 31, 2008

(51) Int. Cl. C12Q 1/68

(52) U.S. Cl. 435/6

(57) ABSTRACT

A method for the detection of all strains of Group B Streptococcus (GBS) (Streptococcus agalactiae) in a mammal comprises isolating nucleic acid from a biological sample obtained from the mammal, detecting in the isolated nucleic acid a specific target region of GBS ssrA gene or tmRNA, an RNA transcript of the GBS ssrA gene, which is indicative of the presence of GBS. The isolated nucleic acid can be contacted with at least one oligonucleotide complementary to the specific target region of the GBS ssrA gene or tmRNA allowing a probe assay to be performed directly on the biological sample. Alternatively, the isolated nucleic acid can be amplified with at least one primer complementary to a specific target region of the GBS ssrA gene or tmRNA in a PCR-based assay. The method according to the invention has the potential to be employed as a screening and/or diagnostic test for use inter alia in hospital laboratories, or as a point-of-care test in various settings where an individual's infection or colonization by GBS is required without delay.

### TECHNICAL FIELD

This invention relates to the detection of Group B Streptococcus (GBS) (Streptococcus agalactiae) in mammals which has application inter alia in detecting the colonization of pregnant women by the organism and consequent risk of transmission to neonates.

### BACKGROUND ART

Group B Streptococcus (GBS) (Streptococcus agalactiae) is one of the leading causes of neonatal morbidity and mortality in the developed world. Early-onset GBS disease occurs within the first week of life and is associated with neonatal sepsis, pneumonia and meningitis.

The mortality rate averages at 6.5% for early-onset GBS cases and for infected preterm infants it rises to 22.7% (Centers for Disease Control and Prevention (2004) Morb. Mortal. Wkly. Rep. 53: 502-505).

Approximately 10-40% of pregnant women carry GBS asymptomatically in their vagina or rectum (Centres for Disease Control and Prevention (2002) Morb. Mortal. Wkly. Rep. 51:1-6; Meyn, L. A. et al (2002), Am. J. Epidemiol. 155: 949-957.

Transmission to the infant occurs vertically during labour via fetal aspiration of infected amniotic fluid or during passage through the birth canal. Since the implementation of intrapartum antibiotic prophylaxis (IAP) for the prevention of GBS, the incidence of early-onset GBS disease in neonates has decreased significantly. Studies in the USA show that the incidence has declined from 1.5/1000 live births in 1990 (Zangwill, K. M et al (1992) Morb. Mortal. Wkly. Rep. 41: 25-32) to 0.32/1000 live births in 2003 (Centers for Disease Control and Prevention (2004) supra). In the UK, early-onset GBS disease is reported to occur in 0.5/1000 births (Royal College of Obstetricians and Gynaecologists (2003) Green-top Guideline No. 36. London: RCOG).

Practices recommended by public health authorities for the identification of at-risk women who should receive antibiotic treatment during labour vary internationally.

Due to the dynamic status of vaginal GBS colonization, screening intrapartum is the most accurate method of predicting the GBS colonization status (Centres for Disease Control and Prevention (2002) supra, Yancey, M. K. et al (1996) Obstet. Gynecol. 88: 811-815).

Although the administration of intrapartum antibiotics has proven to be highly effective in lowering early-onset GBS disease in newborns (Centres for Disease Control and Prevention (2002) supra), the widespread use of antibiotics during labour carries risks for mother and infant such as danger of anaphylaxis, the creation of antibiotic resistance and effects on neonatal immune development (Royal College of Obstetricians and Gynaecologists (2003) supra).

Thus, screening for GBS at the time of delivery allows antibiotics to be used more effectively thereby rationalizing their use and minimizing associated risks. The value of a rapid and accurate test for GBS colonization is especially apparent in cases of premature delivery of <35 weeks gestation when screening results may not be available and babies are at greatest risk of developing early-onset GBS disease.

Centres for Disease Control and Prevention (CDC) guidelines for the prevention of perinatal GBS disease recommend a universal, microbiological culture-based prenatal screening strategy at 35-37 weeks' gestation combined with IAP for GBS-colonized women (Centres for Disease Control and Prevention (2002) Morb. Mortal. Wkly. Rep. 51: 1-6).

Conversely, the risk factor approach relies on the presence of one or more of the following intrapartum factors as indicators for increased risk of neonatal GBS infection: previous infant with early-onset GBS disease, premature labour, prolonged rupture of membranes and fever (Royal College of Obstetricians and Gynaecologists (2003) supra).

A comparison of the two strategies showed that the culture-based approach was almost 50% more effective in preventing early-onset GBS disease (Schrag, S. J et al (2002) N. Engl. J. Med. 347:233-239).

However, the UK-based Royal College of Obstetricians and Gynaecologists (RCOG) opposes the practice of universal screening and widespread use of intrapartum antibiotics due to a lack of clear evidence of the effectiveness of such practices in controlling the incidence of neonatal sepsis (Royal College of Obstetricians and Gynaecologists (2003) supra).

Perinatal GBS disease prevention practices have been successful in lowering neonatal GBS disease by 50-80% (Daley A. J., and. Garland., S. M (2004) J. Paediatr. Child Health, 40: 664-668; Schrag, S. J. et al N. Engl. J. Med. 342: 15-20). Nevertheless, early-onset GBS disease cases continue to occur, leading to acute clinical complications especially for preterm infants (Puopolo, K. M et al (2005) Pediatrics 115:1240-1246).

Another area where detection of and/or screening for GBS is important is in the area of animal husbandry.

GBS is an important pathogen associated with bovine mastitis. Mastitis is an inflammation or an infection of the bovine mammary gland or udder. The presence of mastitis in a herd can have enormous economic consequences for milk producers.

Mastitis can have a substantial impact on quantity and quality of the milk produced. In infected cattle, GBS can survive for long periods within the mammary gland. Undiagnosed infected cattle, which are not selected for treatment, may function as reservoirs of infection.

Current diagnostic methods for identification of bacterial pathogens associated with mastitis rely on culture based isolation of the causative agent from milk samples. Culture methods are slow and they can be insensitive.

Therefore, molecular methods, in particular PCR-based methods that can rapidly identify specific bacterial pathogens causing mastitis, enable rapid diagnosis of the causative agent.

Rapid detection of the causative agent is important for determining the appropriate treatment for the disease.

WO2000/070086 discloses the use of the ssrA gene or tmRNA, an RNA transcript of the ssrA gene, or fragments thereof as target regions in a nucleic acid probe assay for the detection and identification of prokaryotic and/or eukaryotic organisms. Newly identified regions of homology and non-homology of the ssrA gene for different organisms provide the basis of identifying and detecting organisms at the molecular level.

Several reports demonstrate that real-time PCR is a rapid, more sensitive method than standard culture for determining the intrapartum GBS colonization status (Convert M et al. (2005) Clin Microbiol Infect; 11: 1022-1026; Davies H. D et al. (2004) CID 39: 1129-1135; Gavino M., Wang E. (2006) American Journal of Obstetrics & Gynecology 388.e1-e4).

As a result, intrapartum antibiotic prophylaxis can be administered more effectively, thereby reducing the transmission rates of GBS to infants and consequently lowering infant morbidity and mortality rates.

Clinical studies have shown an increase in sensitivity for real-time PCR tests in comparison to the gold standard culture method (Convert M et al. (2005) supra; Uhl J. R et al. (2005) J. Clin. Microb. 43: 4046-4051).

There is a need for a rapid diagnostic test for GBS that can be performed inter alia in a labour ward setting to ascertain the GBS colonization status of women in labour, those in preterm labour or women who have not had prenatal care. For these women culture-screening is not useful because of the time it takes to obtain test results.

There is an on-going need for molecular methods in particular PCR-based methods that can rapidly identify the presence of GBS in various mammals.

In particular, there is a need for methods allowing for improved clinical specificity, and methods allowing for the detection of closely related serotypes of GBS.

Real-time PCR tests are available.

There is also a need for a GBS detection method which can distinguish between live and dead GBS organisms, and therefore determine if the GBS is from a current or past infection.

There is also a need for a rapid diagnostic test for GBS to ascertain the GBS colonization status of cows, so that mastitis caused by GBS may be diagnosed at an early stage to allow for appropriate and effective action to be taken.

### DISCLOSURE OF THE INVENTION

Accordingly, the invention provides a method for the detection of all strains of Group B Streptococcus (GBS) (Streptococcus agalactiae) in a mammal, which method comprises isolating nucleic acid from a biological sample obtained from the mammal, detecting in the isolated nucleic acid a specific target region of GBS ssrA gene or tmRNA, an RNA transcript of the GBS ssrA gene, which is indicative of the presence of GBS.

The method according to the invention, which is carried out in vitro, allows for the detection of GBS with high clinical specificity.

The method according to the invention also allows for the design of assays with a high degree of confidence.

The method according to the invention can involve use of the GBS ssrA gene or tmRNA, an RNA transcript of the GBS ssrA gene, which are present in all bacteria.

Thus, the method according to the invention greatly facilitates the early diagnosis and treatment of GBS.

The method according to the invention allows for samples to be processed with higher speed and efficiency than current culture-based detection methods.

A further advantage of the present invention is that it permits one to diagnose, or aid in the diagnosis of GBS colonization or to otherwise make a negative diagnosis.

According to one embodiment of the invention the isolated nucleic acid is contacted with at least one oligonucleotide complementary to the specific target region of the GBS ssrA gene or tmRNA.

Thus, a probe assay may be performed directly on the biological sample for the presence of GBS.

According to one embodiment of the invention the isolated nucleic acid is amplified with at least one primer complementary to a specific target region of the GBS ssrA gene or tmRNA.

The method according to the invention provides for the detection of low numbers of GBS organisms in a biological sample by amplifying the ssrA gene or tmRNA target region.

According to one embodiment of the invention the biological sample is selected from blood or an oral, mammary gland, nasal, rectal or vaginal secretion.

The method according to the invention also improves on the capability to diagnose, detect and monitor GBS using a reliable and non-invasive technique.

According to one embodiment of the invention, the sample has been obtained by means of a swab.

Advantages of using swabs to collect the biological sample are that they can be sent to the laboratory in transport medium at ambient temperature rather than having to be kept cool, and they are simpler to process than other samples such as urine.

Preferably, the nucleic acid is isolated by lysis of a suspension of presumptive GBS cells by mechanical disruption.

An advantage of using a lysis method based on mechanical disruption of the swab suspension is that it is a simple and rapid technique of isolating the nucleic acid from the sample.

Preferably, the product of amplification is used as a target region for a nucleic acid probe.

The method according to the invention provides for the detection of low numbers of GBS organisms in a biological sample by amplifying the ssrA gene or tmRNA target region before probing with a nucleic acid to indicate its presence.

Preferably, the target region of the GBS ssrA gene is a 293bp sequence as shown in FIG. 1 (SEQ ID NO: 1).

According to one embodiment of the invention the target region of GBS ssrA gene is amplified using GBS specific forward and reverse primers.

No other nucleic assay probe assay has been reported which uses regions of the ssrA gene as a target region to detect the presence of GBS, including colonization of a mammal.

According to a further embodiment of the invention the target region is tmRNA which is reverse transcribed into cDNA before being amplified using GBS forward and reverse primers.

The reverse transcription of tmRNA into cDNA enables the use of the same GBS forward and reverse primers used to amplify the GBS ssrA gene DNA.

According to one embodiment of the invention the specific forward primer is 5′-GACAGGCATTATGAGGTA-3′ (SEQ ID NO: 2).

According to a further embodiment of the invention the specific reverse primer is 5′-GCTAATATATTTGTCTACAAC-3′ (SEQ ID NO: 3).

The forward and reverse primers in accordance with the invention were designed based on Clustal W alignment of the ssrA gene of GBS strains representing a number of the most commonly occurring serotypes and other related streptococci.

Sequence analysis indicated that the GBS ssrA gene was divergent from the other related streptococci, thus facilitating the design of GBS ssrA specific oligonucleotides.

According to one embodiment of the invention the target region of GBS ssrA gene is detected using a pair of GBS specific probes.

The nucleic acid probes in accordance with the invention typically consist of at least 10 nucleotides of the complementary sequence of the GBS ssrA gene and are used to hybridize to the GBS ssrA PCR product.

Probe hybridization to its complementary sequence is typically revealed by detecting a labelled probe.

The label for the probe may be an entity detectable by biochemical, photochemical, immunological, spectroscopic, biophysical or any chemical means.

Preferably, the label is selected from the group consisting of an affinity label, biotin, a chromophore, a colloidal metal, dioxigenin, a dye, an enzyme, an enzyme substrate, a fluorophore, a lumiphore, a magnetic particle, a metabolite, a radioisotope and streptavidin.

Most preferably, the probes are labelled with fluorophore groups.

According to one embodiment of the invention the first probe is 5′-TTGCGTTTTGCTAGAAGGTCTTA-Flu-3′ (SEQ ID NO: 4).

The first probe is labeled at the 3′-end with a fluorescein group.

According to a further embodiment of the invention the second probe is 5′-LC640-TATCAGCAAACTACGTTTGGCT-Ph-3′ (SEQ ID NO: 5).

The second probe is labelled at the 5′-end with a LightCycler® Red fluorophore (LightCycler® Red 640), it is also 3′-phosphorylated so that it cannot be extended.

Further, preferably, the probes interact based on Fluorescence Resonance Energy Transfer (FRET) and indicate the presence of the GBS ssrA DNA or cDNA.

FRET is a process by which transfer of energy occurs from an excited state fluorophore group (donor) to a second fluorophore group (acceptor) when in close proximity. The transferred energy enables the second fluorophore group to emit a fluorescence signal which can be measured on a suitable instrument such as a LightCycler®.

When hybridized to the template DNA or cDNA, the two probes are close enough to allow fluorescence resonance energy transfer (FRET) between the two fluorophores.

According to one embodiment of the invention the method is used to detect the colonization of the mammal by GBS.

Preferably, the mammal is a woman.

Further, preferably, the mammal is a pregnant woman.

According to a further embodiment of the invention the biological sample is a secretion which has been obtained from the genital tract area of the woman.

By providing a method for detecting GBS colonization the method according to the invention will aid in preventing transmission of GBS present in the genital tract area of a woman, to a neonate during labour.

According to one embodiment of the invention, the sample has been obtained by vaginal swab.

According to an alternative embodiment of the invention, the sample has been obtained by rectal swab.

According to a still further embodiment of the invention, the sample has been obtained by vaginorectal swab.

Preferably, the method according to the invention is capable of detecting GBS with a sensitivity higher than 90% relative to culture detection.

Thus, the method according to the invention is able to correctly distinguish between GBS positive and GBS negative samples in over 90% of samples tested, when compared with the culture detection method.

Further, preferably, the method according to the invention is capable of detecting GBS with a sensitivity higher than 95% relative to culture detection.

Thus, the method according to the invention is able to correctly distinguish between GBS positive and GBS negative samples in over 95% of samples tested, when compared with the culture detection method.

The sensitivity of the method according to the invention is higher than the commercially available FDA-approved BD GeneOhm™ StrepB Assay.

Preferably, the method according to the invention is capable of detecting GBS with a specificity greater than 90% relative to culture detection.

Further, preferably the method according to the invention is capable of detecting GBS with a specificity greater than 95% relative to culture detection.

The specificity of the method according to the invention is higher than the commercially available FDA-approved BD GeneOhm™ StrepB Assay.

The method according to the invention is capable of detecting GBS with a specificity of 100% when tested with other related streptococci.

According to one embodiment of the invention the method is capable of detecting GBS colonization in less than 2 hours.

The present invention enables GBS colonization to be detected more rapidly than current culture based detection methods, thereby permitting early diagnosis and medical intervention.

According to a further embodiment of the invention the method is capable of detecting GBS colonization in less than 1 hour 15 minutes.

The method according to the invention is capable of distinguishing between living and dead GBS organisms.

The detection of the short lifespan tmRNA is indicative of a living GBS organism. However GBS ssrA gene DNA, is capable of surviving for a longer period and if detected could be the result of a living or dead organism.

According to a further embodiment of the invention the method is capable of distinguishing between current and past GBS infection.

The method according to the invention provides for the isolation of tmRNA, an RNA transcript of the GBS ssrA gene, followed by treatment with a DNase to remove any contaminating DNA. The detection of the GBS tmRNA is indicative of a current GBS infection. In contrast, the detection of GBS ssrA gene DNA is indicative of a current or a past infection.

Thus, the method according to the invention represents a significant improvement relative to current methods for the detection of and colonization by GBS.

According to one embodiment of the invention there is provided a method of determining the risk of early-onset GBS disease in a neonate, which comprises carrying out a method as hereinbefore defined.

According to a further embodiment of the invention the mammal is a ruminant.

Preferably, the ruminant is a cow.

Preferably, the biological sample is milk.

According to one embodiment of the invention there is provided a method of detecting GBS associated with mastitis.

Thus, the method according to the invention facilitates the early diagnosis and relevant treatment of GBS associated with mastitis, especially as it enables the design of assays which can distinguish between current and past infection.

According to one embodiment of the invention there is provided a method for the detection of colonization by all strains of Group B Streptococcus (GBS) (Streptococcus agalactiae) in a mammal, substantially as hereinbefore described and exemplified.

### BRIEF DESCRIPTION OF THE DRAWINGS

FIG. 1 shows the 293 bp sequence of the GBS ssrA gene (SEQ ID NO: 1) as described in Example 1. The 293 bp PCR product is amplified from the sequence using the primer sequences which are underlined;

FIG. 2 is the ssrA sequence (SEQ ID NO: 12) of GBS serovar 15081_Ia;

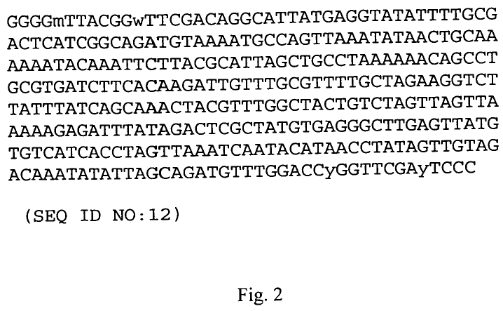

FIG. 3 is the ssrA sequence (SEQ ID NO: 13) of GBS serovar 15082_Ib;

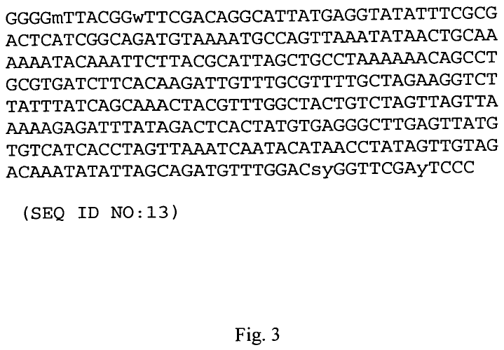

FIG. 4 is the ssrA sequence (SEQ ID NO: 14) of GBS serovar 15083_Ic;

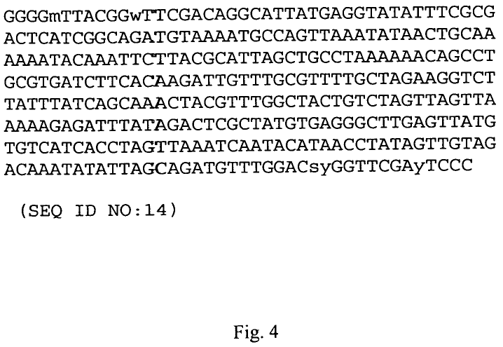

FIG. 5 is the ssrA sequence (SEQ ID NO: 15) of GBS serovar 15084_II;

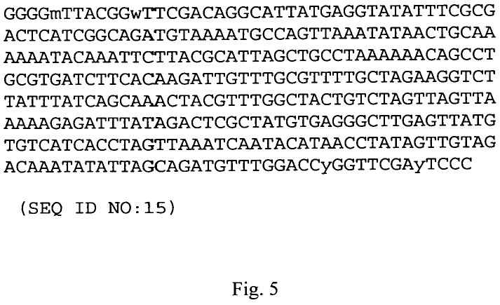

FIG. 6 is the ssrA sequence (SEQ ID NO: 16) of GBS serovar 15085_III;

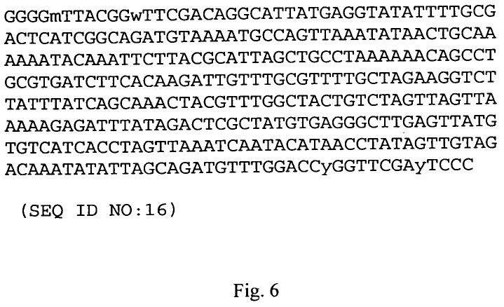

FIG. 7 is the ssrA sequence (SEQ ID NO: 17) of GBS serovar 15086_IV;

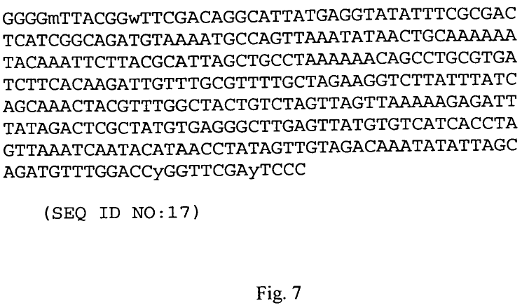

FIG. 8 is the ssrA sequence (SEQ ID NO: 18) of GBS serovar 15087_V;

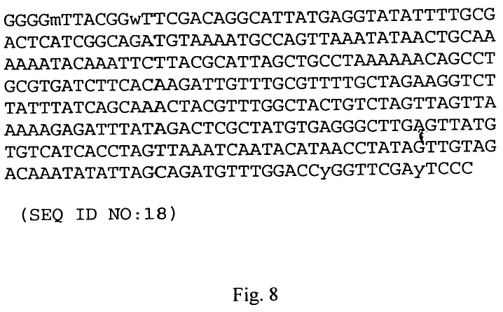

FIG. 9 is the ssrA sequence (SEQ ID NO: 19) of GBS serovar 15090_Ib;

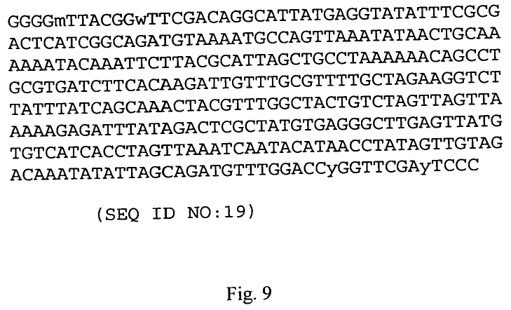

FIG. 10 is the ssrA sequence (SEQ ID NO: 20) of GBS serovar 15094_III; and

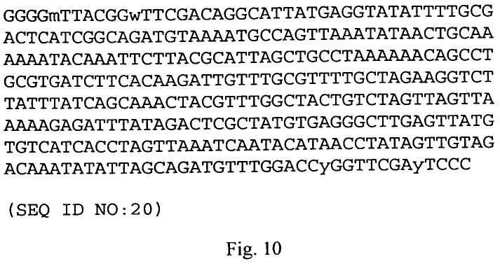

FIG. 11 is the ssrA sequence (SEQ ID NO: 21) of GBS serovar 15095_III.

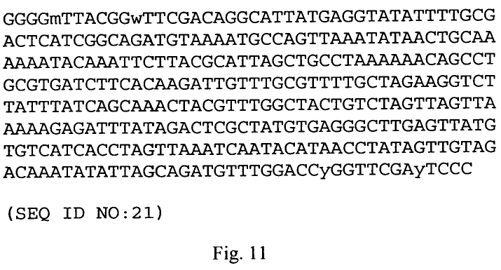

### MODES FOR CARRYING OUT THE INVENTION

The invention will be further illustrated by the following Examples with reference to the detection of GBS colonization in pregnant women.

### EXAMPLE 1

### Collection of Specimens

Vaginal swabs from pregnant women (n=39) were sourced from the Department of Obstetrics and Gynaecology, University College Hospital Galway (UCHG), Ireland. Ethics consent was obtained from the Research Ethics Committee at UCHG. Duplicate vaginal swabs were collected into Amies transport medium (Sarstedt, Nümbrecht, Germany), transported to the laboratory at ambient temperature and stored at 4° C. until required.

Vaginal swab specimens from pregnant women (n=120) were purchased from The New England Life Science Group (NELSG) (Los Osos, Calif., USA), a clinical services organization.

These specimens were remnant swabs screened for GBS colonization by USA hospital laboratories as part of routine prenatal care. GBS was identified in these swabs at source by genital screen cultures or by selective GBS culture.

The remnant specimens were frozen within 1-3 days of sampling and shipped on dry ice to our laboratory. A proportion of 90% GBS-positive specimens (n=107) was requested from NELSG. Those undertaking the performance evaluation of the nucleic acid tests were blinded to the results of the US microbiological analysis while the test was in progress.

### Culture

One of the duplicate antenatal specimens collected from UCHG was used for microbiological evaluation. The swab was inoculated into 7 ml LIM broth (Todd-Hewitt broth with 15 μg/ml nalidixic acid and 10 μg/ml colistin) (LIP Diagnostic Services, Galway, Ireland), incubated overnight and subcultured onto Trypticase Soy Agar (TSA)+5% sheep blood agar (LIP) for 18-24 h. at 37° C. according to CDC recommendations for GBS culture processing.

Presumptive GBS colonies showing β-haemolysis were confirmed using catalase test and antigen detection (Streptococcal Grouping kit, Oxoid, Cambridge, UK). Plates showing no colony growth were reincubated for a further 24 hours and reinspected.

Remnant specimens collected from US sites were screened at source for GBS using the CDC-recommended method described above or by genital culture including LIM broth culture.

### Sample Preparation

The commercially available crude lysis kit BD GeneOhm™ Lysis Kit (BD, NJ, USA) was used for sample preparation. The second swab of duplicate UCHG specimens and remnant swabs were vortexed for 2 min in 1 ml of sample buffer. Four hundred microlitres of the suspension was transferred into the lysis tube and lysed by mechanical disruption with silica beads according to manufacturer's instructions. Lysates were stored at −20° C. until required.

### Construction of an Internal Amplification Control (IAC)

The inclusion of an internal amplification control (IAC) in PCR reactions serves to identify false negative results caused by malfunction of the thermal cycler, incorrect PCR mixture, poor DNA polymerase activity or the presence of inhibitors in the reaction (Hoorfar, J. B. et al. (2004) J. Clin. Microbiol. 42:1863-1868).

An internal amplification control (IAC) was constructed using the composite primer approach described by Hoorfar et al. ((2004) supra). An internal amplification control consisting of heterologous DNA cloned into a plasmid vector was included in the test in accordance with the invention to identify false negative test results caused by PCR inhibition. The IAC was co-amplified with the GBS target and detected by an IAC-specific hybridization probe included in the real-time PCR reaction. The IAC specific primers (SEQ ID NOS: 6 and 7) and probes (SEQ ID NOS: 10 and 11) used are included in Table 1.

### GBS Real-Time PCR Test According to the Invention

Real-time PCR was performed in a 20 μl reaction volume on the LightCycler® instrument using the “LightCycler® FastStart DNA Master HybProbe” kit (Roche Diagnostics, Mannheim, Germany). Each reaction contained reagents to final concentrations of: 5 mM MgCl₂, 0.5 μM of each primer (SEQ ID NOS: 2 and 3) (Table 1), 0.2 μM of each hybridization probe (SEQ ID NOS: 4 and 5) (Table 1) and 0.5 U uracil-DNA glycosylase (Roche).

TABLE 1

Oligonucleotide primers and
hybridization probes used

| | | |
|---|---|---|
| Name | Function | Sequence 5′-3′ |

| | | |
|---|---|---|
| gbsU3F | Forward primer | GACAGGCATTATGAGGTA |
| | | (SEQ ID NO: 2) |
| gbsU4R | Reverse primer | GCTAATATATTTGTCTACAAC |
| | | (SEQ ID NO: 3) |
| CompF | Forward composite | GACAGGCATTATGAGGTAATA |
| | primer for IAC | CCCAACTTGGAATG |
| | generation | (SEQ ID NO: 6) |
| CompR | Reverse composite | GCTAATATATTTGTCTACAAC |
| | primer for IAC | TCTTCACCAGAATAAAATTG |
| | generation | (SEQ ID NO: 7) |
| tmUF | Universal bacterial | GGGG(A/C)(C/T)TACGG |
| | ssrA forward primer | (A/T)TTCGAC |
| | | (SEQ ID NO: 8) |
| tmUR | Universal bacterial | GGGA(A/G)TCGAACC(A/G) |
| | ssrA reverse primer | (C/G)GTCC |
| | | (SEQ ID NO: 9) |
| f1GBS-Flu | GBS-specific | TTGCGTTTTGCTAGAAGGTCT |
| | hybridization probe | TA-Flu |
| | | (SEQ ID NO: 4) |
| f2GBS- | GBS-specific | LC640-TATCAGCAAACTACG |
| LC640 | hybridization probe | TTTGGCT-Ph |
| | | (SEQ ID NO: 5) |
| ALS1-Flu | IAC-specific | TGAATGTATCCCCTGGA-Flu |
| | hybridization probe | (SEQ ID NO: 10) |
| ALS1- | IAC-specific | LC705-TGGCACTGGTACCAT |
| LC705 | hybridization probe | CTAA-Ph |
| | | (SEQ ID NO: 11) |

Template was added in 2 μl volumes and IAC was added as 100 recombinant plasmid copies per reaction. Thermal cycling parameters consisted of 95° C. denaturation for 10 min followed by 50 amplification cycles of 95° C. for 10 s, 50° C. for 15 s and 72° C. for 10 s. Melting profiles were run between 40° C. and 80° C. at a transition rate of 0.1° C./s.

The performance of the GBS real-time PCR test for the detection of GBS in clinical samples in accordance with the invention was benchmarked against the performance of the commercially available FDA-approved BD GeneOhm™ StrepB Assay.

The BD GeneOhm™ StrepB assay was performed on the SmartCycler® instrument according to manufacturer's instructions.

Briefly, 25 μl of diluent was added to each Master Mix reaction tube. Positive and negative control tubes were included in each run. Sample crude lysates were added to the Master Mix tubes in 1.5 μl volumes, the tubes were briefly centrifuged and placed in the SmartCycler® instrument. Assay runs were performed using the BD GeneOhm™ StrepB Assay-specific software.

### Results

### Real-Time PCR Assay Design

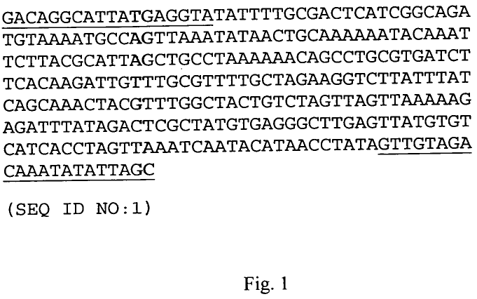

The ssrA genes of ten GBS strains representing seven of the most commonly occurring serotypes were sequenced (Sequiserve, Vaterstetten, Germany) and aligned with other related streptococci (see Table 2) ssrA sequences generated in this Example or available on the tmRNA website (Williams., K. P. (2000) Nucleic Acids Res. 28: 168-170) (Table 1). From these alignments, oligonucleotide primers gbsU3F (SEQ ID NO: 2) and gbsU4R (SEQ ID NO: 3) were designed to amplify a 293 bp (SEQ ID NO: 1) PCR product from the GBS ssrA gene. The sequence of the 293 bp (SEQ ID NO: 1) PCR product is shown in FIG. 1.

A fluorescently labelled hybridization probe pair (f1GBS-Flu (SEQ ID NO: 4) and f2GBS-LC640 (SEQ ID NO: 5)) was designed for the detection of GBS. BLAST (Basic Local Alignment Search Tool) analysis of the hybridization probe sequences was performed to confirm in silico specificity of the probes for the detection of GBS.

TABLE 2
Bacterial species and strains used to test the specificity of the real-
time PCR test in accordance with the invention.

| | Collection ref |
|---|---|
| | no |
| GBS | |
| Streptococcus agalactiae | BCCM 15081 |
| Streptococcus agalactiae | BCCM 15082 |
| Streptococcus agalactiae | BCCM 15083 |
| Streptococcus agalactiae | BCCM 15084 |
| Streptococcus agalactiae | BCCM 15085 |
| Streptococcus agalactiae | BCCM 15086 |
| Streptococcus agalactiae | BCCM 15087 |
| Streptococcus agalactiae | BCCM 15090 |
| Streptococcus agalactiae | BCCM 15094 |
| Streptococcus agalactiae | BCCM 15095 |
| Strep spp | |
| Streptococcus dysgalactiae | DSM 6176 |
| Streptococcus oralis | DSM 20627 |
| Streptococcus pneumoniae | DSM 11865 |
| Streptococcus parasanguinis | DSM 6778 |
| Streptococcus intermedius | DSM 20573 |
| Streptococcus salivarius | DSM 20560 |
| Streptococcus uberis | DSM 20569 |
| Streptococcus mitis | DSM 12643 |
| Enterococcus faecalis | DSM 20371 |
| Enterococcus faecium | DSM 20477 |
| Streptococcus gordonii | DSM 6777 |
| Streptococcus mutans | DSM 20523 |
| Streptococcus pyogenes | DSM 20565 |
| Streptococcus angiosus | |
| Genital Panel | |
| Acintobacter lwoffii | DSM 2403 |
| Lactobacillus acidophilus | DSM 20079 |
| Lactobacillus fermentum | DSM 20055 |
| Mobiluncus curtisii | DSM 2711 |
| Mobiluncus mulieris | DSM 2710 |
| Stool Panel | |
| Escherichia coli | DSM 30038 |
| Morganella morganii subsp morganii | DSM 30164 |
| Proteus mirabilis | DSM 4479 |
| Klebsiella pneumoniae | Clinical isolate |
| Escherichia hermanii | DSM 4560 |
| Escherichia vulneris | DSM 4564 |
| Aeromonas hydrophilia | DSM 30015 |
| Citrobacter freundii | DSM 30039 |
| Enterobacter cloacae | ATCC 13047 |
| Respiratory Panel | |
| Staphylococcus aureus | DSM 11965 |
| Staphylococcus epidermidis | ATCC 27626 |
| Pseudomonas aeruginosa | ATCC 10145 |
| Klebsiella oxytoca | DSM 5175 |
| Neisseria Panel | |
| Neisseria meningitidis | DSM 10036 |
| Neisseria lactamica | DSM 4691 |
| Neisseria meningitidis | ATCC 13077 |
| Neisseria cinera | DSM 4630 |
| Sepsis Panel | |
| Streptococcus sanguis | DSM 20567 |
| Streptococcus porcinus | DSM 20725 |
| Serratia marcescens | DSM 1608 |
| Enterobacter aeruginosa | DSM 30053 |
| Staphylococcus haemolyticus | DSM 20263 |
| Streptococcus bovis | DSM 20480 |

### Analytical Sensitivity and Specificity of the GBS Real-Time PCR Test According to the Invention

The limit of detection (LOD) was established using crude lysate extracted from serial dilutions of overnight GBS culture (BCCM 15081) using the IDI Lysis kit (GeneOhm Sciences, Canada). Colony forming units (cfu) per dilution were established by triplicate plate counts and real-time PCR reactions included GBS crude lysate template containing between 10⁵ and 10⁻¹ cell equivalents.

The LOD was determined by three independent experimental assessments and the GBS test according to the invention consistently detected in the range of 1-10 cell equivalents per reaction. The inclusion of internal amplification control at a concentration of 100 plasmid copies per test did not lower the LOD (data not shown). At high GBS template concentrations (≧10⁵ cell equivalents/reaction) the amplification of internal control target was occasionally inhibited due to competition between GBS target DNA and IAC in the PCR reaction.

Specificity studies were performed using DNA prepared from a panel of ten GBS strains representing seven of the most commonly occurring serotypes and forty two related streptococci and other species found in the genital tract environment as shown in Table 2.

All ten GBS strains were detected. The test was negative for all non-GBS species listed in Table 2. The amplifiability of DNA extracted from non-GBS species was confirmed with universal primers tmUF (SEQ ID NO: 8) and tmUR (SEQ ID NO: 9) listed in Table 1 and which were designed to amplify the bacterial ssrA gene.

### Evaluation of the GBS Test According to the Invention in Clinical Samples

Performance of the GBS test according to the invention was compared to the results of microbiological culture methods for the isolation of GBS. Test results were also compared to those obtained using the commercial BD GeneOhm™ StrepB diagnostic test. Microbiological culture results were used as the gold standard.

A total of 159 specimens (39 from UCHG, 120 from NELSG) were tested by microbiological culture, the GBS real-time PCR test according to the invention and BD GeneOhm™ StrepB Assay. Microbiological culture identified 111 samples as GBS-positive and 48 samples as GBS-negative.

The prevalence of GBS colonization among the specimens collected from UCHG was 10.3% (4/39) using the standard culture method. The relatively low carriage rate may be explained by the fact that the swabs were vaginal rather than recto-vaginal samples, the gut being a natural reservoir of GBS (Badri M. S et al (1977) J. Infect. Dis. 135: 308-312).

Calculation of prevalence of GBS colonization in the NELSG samples is not applicable since this population of specimens was selected to be predominantly GBS-positive and is not representative of the true prevalence among pregnant women.

In 146 samples, the results of all three tests showed 100% correlation, where 104 samples were identified as GBS-positive and 42 samples were identified as GBS-negative. In 95% of samples the two nucleic acid diagnostic (NAD) test results agreed (151/159).

Discrepant results were obtained in a total of 13 samples after retesting the crude lysate. In 5 samples, NAD test results agreed but conflicted with the microbiology results. Microbiological culture identified 2 samples as GBS-negative which the NAD tests identified as GBS-positive. Three samples were determined as GBS-positive by culture and tested negative in the NAD tests.

In one sample, the GBS test according to the invention was negative for a sample that was GBS-positive by both culture and BD GeneOhm™ StrepB test. PCR inhibition was not apparent since the IAC gave a positive signal.

For 6 samples, the BD GeneOhm™ StrepB test results disagreed with concurring culture and GBS real-time PCR results (3 false positives, 3 false negatives).

In another sample, the results of both the IAC and the GBS test according to the invention were negative, indicating PCR inhibition. This specimen was identified as GBS-negative by both culture and BD GeneOhm™ StrepB test with no indication of PCR inhibition. However, the discrepancy was resolved for this sample by increasing the IAC to 200 plasmid copies in the GBS real-time PCR test according to the invention which yielded a positive result for the IAC for this sample.

Table 3 shows the sensitivity, specificity, predictive values and likelihood ratios for the GBS test according to the invention and the BD GeneOhm™ StrepB test using microbiological culture for the identification of GBS as the gold standard. Confidence intervals (CI) are stated at the 95% level.

Table 3 shows the sensitivity and specificity achieved with the GBS test according to the invention, in comparison to culture was 96.4% (CI 95% 90.5-98.8) and 95.8% (CI 95% 84.6-99.3), respectively.

TABLE 3
Sensitivity, specificity, predictive values (PPV, NPV) and
likelihood ratios +LR, −LR) for the GBS test and
the BD GeneOhm ™ StrepB Assay.

| | | | | BD GeneOhm ™ StrepB | |
|---|---|---|---|---|---|
| | | GBS test | | Assay | |
| | | Positive | Negative | Positive | Negative |
| Culture^{a} | Positive | 107 | 4 | 105 | 6 |
| | Negative | 2 | 46 | 5 | 43 |

| | | |
|---|---|---|
| Sensitivity | 96.4% | 94.6% |
| 95% CI | (90.5-98.8%) | (88.1-97.8%) |
| Specificity | 95.8% | 89.6% |
| 95% CI | (84.6-99.3%) | (76.6-96.1%) |
| PPV^{b} | 98.2% | 95.5% |
| 95% CI | (92.9-99.7%) | (89.2-98.3%) |
| NPV^{b} | 92% | 87.8% |
| 95% CI | (79.9-97.4%) | (74.5-94.9%) |
| +LR^{b} | 23.1 | 9.1 |
| 95% CI | (5.9-89.9) | (3.9-20.8) |
| −LR^{b} | 0.04 | 0.06 |
| 95% CI | (0.01-0.1) | (0.03-0.13) |

^{a}using culture as the gold standard (n = 159)
^{b}sample population comprised of random (n = 39) and non-random (n = 120) specimens

### EXAMPLE 2

Use of GBS tmRNA in an RNA-Based Assay

A two-step assay with an independent RT step was carried out using primer gbsU4R (SEQ ID NO: 3) followed by real-time PCR using gbsU3F (SEQ ID NO: 2)/gbsU4R (SEQ ID NO: 3) primers and FRET1/2 hybridization probe pair (SEQ ID NO: 4/5). The performance of this assay was evaluated using serial dilutions (10⁹-10⁻¹) of GBS cells from which RNA was extracted using the Ambion RNA kit. In parallel, crude lysates from serial dilutions (10⁹-10⁻¹) of GBS cells were generated using the IDI lysis kit (GeneOhm Sciences, Canada).

The performance of each method was assessed by including the extracted/released RNA in a GBS RT-real-time PCR assay as hereinabove described. The same limit of detection was achieved with both methods enabling 1-10 GBS cell equivalents to be detected.

The qualitative real-time PCR test according to the invention provides for the rapid detection of GBS (75 min. including sample preparation) and is capable of detecting 1-10 genome copies of GBS.

As indicated in Example 1 sensitivity and specificity achieved with the test in comparison to culture was 96.4% and 95.8%, respectively.

The GBS real-time PCR test performed better than the commercial FDA-approved BD GeneOhm™ Strep B Assay (sensitivity 94.6% (CI 95% 88.1-97.8), specificity 89.6% (CI 95% 76.6-96.1)).

Test results from the GBS real-time PCR test and BD GeneOhm™ Strep B Assay correlated in 95% of samples. In five samples the culture results disagreed with concurring NAD test results. Two of these five samples were identified as culture-negative but GBS-positive by both real-time PCR tests. The higher detection rates of the real-time PCR tests may be explained by the inability of culture to detect low numbers of organisms, the presence of antagonistic organisms, or possibly the detection of non-viable cells.

Two previous studies evaluated the clinical performance of the BD GeneOhm™ Strep B Assay and reported a sensitivity of 94% and a specificity of 95.9% for direct detection (Davies H. D et al. (2004) CID 39: 1129-1135) and a sensitivity of 92.5% and a specificity of 92.5% after 4 h selective enrichment (Goodrich J. S., and Miller M. B. (2007) Diagnostic Microbiology and Infectious Disease 59 17-22).

Test results from the real-time PCR tests in accordance with the invention correlated in 95% of samples. In five samples the culture results disagreed with concurring NAD test results. Two of these 5 samples were identified as culture-negative but GBS-positive by both real-time PCR tests.

Increased sensitivities for real-time PCR diagnostic tests over the standard culture method have been reported in other studies (Convert M et al. (2005) supra; Davies H. D et al. (2004) supra; Schrag, S. J. et al. (2002) Active Bacterial Core Surveillance Team) and can be a result of detection of non-viable cells, low bacterial burden (Convert M et al. (2005) supra; Goodrich J. S., and Miller M. B. (2007) supra) or the presence of antagonistic microorganisms which inhibit growth in culture (Dunne W. M. Jr., and Holland-Staley, C. A. (1998) J. Clin. Microbiol. 36: 2298-2300. Ostroff, R. M., and Steaffens, J. W. (1995) Diagn. Microbiol. Infect. Dis. 22:253-259).

In three cases, the PCR tests were negative for culture-positive samples. However, this discrepancy may be due to misidentification of GBS during culture screening. Two of these specimens were screened by genital culture and GBS growth may have been misidentified. The third sample was a UCHG duplicate swab in which the DNA may have been degraded. This sample had been stored at 4° C. for 5 days before processing for real-time PCR.

The efficiency of a real-time PCR test is related to the efficiency of the sample preparation method employed. In this Example, a crude lysis method (BD GeneOhm™ Lysis Kit) was chosen over a DNA purification method. The crude lysis method based on mechanical disruption of the swab suspension proved to be simple and rapid (15 min). Furthermore, using this sample preparation method, a 100-fold higher analytical sensitivity was achieved compared to a bacterial DNA purification method (data not shown) indicating either inefficient lysis or PCR inhibition when using the DNA purification method. These findings are in accordance with a previous study (Ke D et al. (2000) Clin. Chem. 46:3 324-331).

One sample showed a negative result for the GBS test according to the invention for both GBS detection and IAC. However, when the test was repeated using double the IAC concentration, the IAC gave a positive result.

The method according to the invention targets the bacterial ssrA gene or tmRNA, an RNA transcript of the ssrA gene in GBS which is a suitable and versatile diagnostic target for GBS as hereinabove described.

The method according to the invention has the potential to be employed as a screening and/or diagnostic test for use inter alia in hospital laboratories, or as a point-of-care test in various settings where an individual's infection or colonization by GBS is required without delay.

What we claim is:

1. A method for the detection of all strains of Group B Streptococcus (GBS) (Streptococcus agalactiae) in a mammal, which method comprises isolating nucleic acid from a biological sample obtained from the mammal, detecting in the isolated nucleic acid a specific target region of GBS ssrA gene or tmRNA, an RNA transcript of the GBS ssrA gene, which is indicative of the presence of GBS.

2. A method according to claim 1, wherein the isolated nucleic acid is contacted with at least one oligonucleotide complementary to the specific target region of the GBS ssrA gene or tmRNA.

3. A method according to claim 1, wherein the isolated nucleic acid is amplified with at least one primer complementary to a specific target region of the GBS ssrA gene or tmRNA.

4. A method according to claim 1 or 2, wherein the biological sample is selected from blood or an oral, mammary gland, nasal, rectal or vaginal secretion.

5. A method according to claim 1 or 2, wherein the sample has been obtained by means of a swab.

6. A method according to claim 1 or 2, wherein the nucleic acid is isolated by lysis of a suspension of presumptive GBS cells by mechanical disruption.

7. A method according to claims 3, wherein the product of amplification is used as a target region for a nucleic acid probe.

8. A method according to claim 7, wherein the target region is a 293bp sequence of the GBS ssrA gene as shown in FIG. 1 (SEQ ID NO: 1).

9. A method according to claim 8, wherein the target region of the GBS ssrA gene is amplified using GBS forward and reverse primers.

10. A method according to claim 3, wherein the target region is tmRNA, which is reverse transcribed into cDNA before being amplified using GBS forward and reverse primers.

11. A method according to claim 9, wherein the forward primer is 5′-GACAGGCATTATGAGGTA-3′ (SEQ ID NO: 2).

12. A method according to claim 9, wherein the reverse primer is 5′-GCTAATATATTTGTCTACAAC-3′ (SEQ ID NO: 3).

13. A method according to claim 3, wherein the target region is detected using a pair of GBS specific probes.

14. A method according to claim 13, wherein the probes are labelled with fluorophore groups.

15. A method according to claim 13, wherein a first probe is 5′-TTGCGTTTTGCTAGAAGGTCTTA-Flu-3′ (SEQ ID NO: 4).

16. A method according to claim 13, wherein a second probe is 5′-LC640-TATCAGCAAACTACGTTTGGCT-Ph-3′ (SEQ ID NO: 5).

17. A method according to claim 13, wherein the probes interact based on Fluorescence Resonance Energy Transfer and indicate the presence of the target region.

18. A method according to claim 1, wherein the method is used to detect the colonization of the mammal by GBS.

19. A method according to claim 18, wherein the mammal is a woman.

20. A method according to claim 18, wherein the mammal is a pregnant woman.

21. A method according to claim 20, wherein the biological sample is a secretion which has been obtained from the genital tract area of the woman.

22. A method according to claim 21, wherein the sample has been obtained by vaginal swab.

23. A method according to claim 21, wherein the sample has been obtained by rectal swab.

24. A method according to claim 21, wherein the sample has been obtained by vaginorectal swab.

25. A method according to claim 18, which is capable of detecting GBS with a sensitivity higher than 90% relative to culture detection.

26. A method according to claim 18, which is capable of detecting GBS with a sensitivity higher than 95% relative to culture detection.

27. A method according to claim 18, which is capable of detecting GBS with a specificity greater than 90% relative to culture detection.

28. A method according to claim 18, which is capable of detecting GBS with a specificity greater than 95% relative to culture detection.

29. A method according to claim 18, which is capable of detecting GBS in less than 2 hours.

30. A method according to claim 18, which is capable of detecting GBS in less than 1 hour 15 minutes.

31. A method according to claim 1 or 3, which is capable of quantitatively distinguishing between living and dead GBS organisms.

32. A method according to claim 1 or 3, which is capable of distinguishing between current and past GBS infection.

33. A method of determining the risk of early-onset GBS disease in a neonate, which comprises carrying out a method according to claim 1 or 3.

34. A method according to claim 1 or 3, wherein the mammal is a ruminant.

35. A method according to claim 34, wherein the ruminant is a cow.

36. A method according to claim 34, wherein the biological sample is milk.

37. A method according to claim 34, for use in detecting GBS associated with mastitis.

38. A method for the detection of all strains of Group B Streptococcus (GBS) (Streptococcus agalactiae) in a mammal, which method comprises isolating a cell lysate or a tissue lysate from a biological sample obtained from the mammal, detecting in the lysate a specific target region of GBS ssrA gene or tmRNA, an RNA transcript of the GBS ssrA gene, which is indicative of the presence of GBS, such detection being by means of nucleic acid amplification followed by direct detection or indirect detection.

39. A method for the detection of all strains of Group B Streptococcus (GBS) (Streptococcus agalactiae) in a mammal, which method comprises isolating whole cells from a biological sample obtained from the mammal, detecting in the whole cells a specific target region of GBS ssrA gene or tmRNA, an RNA transcript of the GBS ssrA gene, which is indicative of the presence of GBS, such detection being by means of nucleic acid amplification followed by direct detection or indirect detection.
